(19) United States
(12) Patent Application Publication
(10) Pub. No.: US 2025/0195584 A1
(43) Pub. Date: Jun. 19, 2025

(54) PHARMACEUTICAL AND COSMETIC AND METHOD FOR PROVIDING SAME

(71) Applicants: Koji TANABE, Palo Alto, CA (US); I Peace, Inc., Palo Alto, CA (US)

(72) Inventor: Koji TANABE, Palo Alto, CA (US)

(73) Assignees: Koji TANABE, Palo Alto, CA (US); I Peace, Inc., Palo Alto, CA (US)

(21) Appl. No.: 18/847,168

(22) PCT Filed: Feb. 3, 2023
(86) PCT No.: PCT/JP2023/003573

§ 371 Date: Sep. 13, 2024

Related U.S. Application Data
(60) Provisional application No. 63/269,378, filed on Mar. 15, 2022.

(51) Int. Cl. A61K 35/545; A61K 8/98; A61P 17/00; A61Q 19/00; A61Q 19/08

(52) U.S. Cl. CPC A61K 35/545; A61K 8/98; A61P 17/00; A61Q 19/007; A61Q 19/08

(57) ABSTRACT

The present invention provides a method for providing a cosmetic or a pharmaceutical comprising obtaining a supernatant of a medium of pluripotent stem cells derived from cells donated by a subject and providing a cosmetic or a pharmaceutical comprising the supernatant of the medium to the subject. The present invention also provides a cosmetic or a pharmaceutical comprising a supernatant of a medium of pluripotent stem cells derived from cells donated by a subject, wherein the cosmetic or the pharmaceutical is provided to the subject.

### TECHNICAL FIELD

The present invention relates to a pharmaceutical and a cosmetic, and a method for providing the same.

### BACKGROUND ART

Embryonic stem cells (ES cells) are stem cells established from an early embryo of a human or a mouse. ES cells have pluripotency, which is a capacity to differentiate into all cells in an organism. Currently, human ES cells can be utilized in cell transplantation therapies for plural diseases, such as Parkinson's disease, juvenile diabetes mellitus, and leukemia. However, transplantation of ES cells has some drawbacks. In particular, transplantation of ES cells can elicit an immune rejection reaction similar to a rejection reaction following unsuccessful organ transplantation. Additionally, there are many critical and opposing views from ethical viewpoints about utilization of ES cells established by destroying human embryos.

Under such circumstances, Professor Shinya Yamanaka of Kyoto University succeeded in establishment of induced pluripotent stem cells (iPS cells) by introducing four types of genes, Oct3/4, Klf4, c-Myc, and Sox2, into somatic cells. This achievement won Professor Yamanaka the 2012 Nobel Prize in Physiology or Medicine (see, for example, Patent Literature 1). iPS cells are ideal pluripotent cells which are free from rejection reaction or ethical issues. Therefore, iPS cells are expected to be used for cell transplantation therapies. Meanwhile, there have been reports showing that a medium used to culture iPS cells was reused as a pharmaceutical composition (see, for example, Patent Literature 2).

### CITATION LIST

### Patent Literature

Patent Literature 1: Japanese Patent No. 4183742
Patent Literature 2: Japanese Patent Laid-open No. 2016-128396

### SUMMARY OF INVENTION

### Technical Problem

The present inventors verified the culture method of Patent Literature 2. As a result, it was found that iPS cells were differentiated by this method, and accordingly, it was appeared that a medium in which differentiated cells, not iPS cells, had been cultured was actually reused. One object of the present invention is to provide a pharmaceutical and a cosmetic obtained by effectively utilizing a medium of iPS cells, and a method for providing the same.

### Solution to Problem

According to an aspect of the present invention, the present invention provides a method for providing a cosmetic or a pharmaceutical, the method comprising providing to a subject a cosmetic or a pharmaceutical comprising a supernatant of a medium of pluripotent stem cells derived from cells donated by the subject. The subject who donates cells and the subject to whom a cosmetic or a pharmaceutical is provided are the same human. Therefore, pluripotent stem cells are autologous cells of the subject to whom the cosmetic or the pharmaceutical is provided.

In the above-described method for providing a cosmetic or a pharmaceutical, the medium may be a medium of pluripotent stem cells cultured and maintained in an undifferentiated state. The medium may also be a medium in which pluripotent stem cells are expressing an undifferentiation marker. The medium may also be a medium of pluripotent stem cells grown in adherent culture. The medium may also be a medium of pluripotent stem cells grown in suspension culture. The medium may also be a medium of pluripotent stem cells grown in three-dimensional stirring culture. The medium may also be a medium of pluripotent stem cells grown in three-dimensional culture without stirring. The medium may also be a gel medium. The medium may contain a deacylated gellan gum or a gellan gum.

The above-described method for providing a cosmetic or a pharmaceutical may further comprise examining the effect of a supernatant of a medium of pluripotent stem cells on somatic cells derived from a subject. A subject from whom somatic cells are derived and a subject from whom pluripotent stem cells are derived may be the same human. Somatic cells may be skin cells.

In the above-described method for providing a cosmetic or a pharmaceutical, the cosmetic or the pharmaceutical may be used for any of retention of skin moisture, amelioration of wrinkles, amelioration of blemishes, amelioration of skin turgor, and amelioration of sagging. The cosmetic or the pharmaceutical may be an injection.

According to an aspect of the present invention, the present invention provides a makeup or treatment method comprising applying or administering to a subject a cosmetic or a pharmaceutical comprising a supernatant of a medium of pluripotent stem cells derived from cells donated by the subject. A subject who donates cells and a subject who receives application or administration of a cosmetic or a pharmaceutical are the same human. Therefore, pluripotent stem cells are autologous cells of the subject to whom the cosmetic or the pharmaceutical is provided.

In the above-described makeup or treatment method, the medium may be a medium in which pluripotent stem cells have been cultured and maintained in an undifferentiated state. The medium may be a medium of pluripotent stem cells grown in adherent culture. The medium may be a medium of pluripotent stem cells grown in suspension culture. The medium may be a medium of pluripotent stem cells grown in three-dimensional stirring culture. The medium may be a medium of pluripotent stem cells grown in three-dimensional culture without stirring. The medium may be a gel medium. The medium may contain a deacylated gellan gum or a gellan gum.

In the above-described makeup or treatment method, the cosmetic or the pharmaceutical may be used for any of retention of skin moisture, amelioration of wrinkles, amelioration of blemishes, amelioration of skin turgor, and amelioration of sagging. The cosmetic or the pharmaceutical may be an injection.

According to an aspect of the present invention, the present invention provides a cosmetic or a pharmaceutical which comprises a supernatant of a medium of pluripotent stem cells derived from cells donated by a subject and is to be provided to the subject. A subject who donates cells and a subject to whom a cosmetic or a pharmaceutical is provided are the same human. Therefore, pluripotent stem cells are autologous cells of the subject to whom the cosmetic or the pharmaceutical is provided.

In the above-described cosmetic or pharmaceutical, the medium may be a medium of pluripotent stem cells cultured and maintained in an undifferentiated state. The medium may be a medium in which pluripotent stem cells are expressing an undifferentiation marker. The medium may be a medium of pluripotent stem cells grown in adherent culture. The medium may be a medium of pluripotent stem cells grown in suspension culture. The medium may be a medium of pluripotent stem cells grown in three-dimensional stirring culture. The medium may be a medium of pluripotent stem cells grown in three-dimensional culture without stirring. The medium may be a gel medium. The medium may contain a deacylated gellan gum or a gellan gum.

In the above-described cosmetic or pharmaceutical, the effect of the supernatant of the medium of pluripotent stem cells on somatic cells derived from a subject may have been examined. The subject from whom somatic cells are derived and the subject from whom pluripotent stem cells are derived may be the same human. Somatic cells may be skin cells.

The above-described cosmetic or pharmaceutical may be used for any of retention of skin moisture, amelioration of wrinkles, amelioration of blemishes, amelioration of skin turgor, and amelioration of sagging. The cosmetic or the pharmaceutical may be an injection.

The cosmetic or the pharmaceutical may be at least one selected from the group consisting of an agent for preventing formation of and ameliorating any of skin blemishes, wrinkles, and sagging, an agent for promoting production of collagen, an agent for promoting production of hyaluronic acid, an agent for promoting dermal/epidermal cell growth, an agent for promoting production of fibroblast growth factor (FGF) family, an agent for promoting production of vascular endothelial growth factor (VEGF), a cytoprotective agent for protecting cells from stresses, a cell survival rate improving agent for improving the survival rate of cells suffering stresses, and a biomaterial protecting agent for protecting an biomaterial from stresses. The FGF family may include FGF-2 and FGF-7. The biomaterial may be at least one selected from the group consisting of nucleic acids, proteins, protein composites, lipoproteins, ribosomes, and biomembranes. The pharmaceutical may be a wound treating agent.

The supernatant of the medium of pluripotent stem cells may have been examined for whether it has at least one effect selected from the group consisting of the effect of preventing formation of and ameliorating skin blemishes, wrinkles, and sagging, the effect of promoting production of collagen, the effect of promoting production of hyaluronic acid, the effect of promoting dermal/epidermal cell growth, the effect of promoting production of fibroblast growth factor (FGF) family, the effect of promoting production of vascular endothelial growth factor (VEGF), the cytoprotective effect of protecting cells from stress, the cell survival rate improving effect of improving the survival rate of cells suffering stress, and the wound treatment effect on somatic cells derived from the subject.

### Advantageous Effects of Invention

According to the present invention, a pharmaceutical and a cosmetic obtained by effectively utilizing a medium of iPS cells and a method for providing the same can be provided.

### BRIEF DESCRIPTION OF DRAWINGS

FIG. 1 is a graph showing the results of a collagen production test of fibroblasts of Example 6.

FIG. 2 is a graph showing the results of a collagen production test of fibroblasts of Example 7.

FIG. 3 is a graph showing the treatment effect of hydro-injection of Example 8.

FIG. 4 is a graph showing the results of a proliferation test of fibroblasts of Reference Example 4.

FIG. 5 is a graph showing the results of a proliferation test of fibroblasts of Reference Example 4.

FIG. 6 is a graph showing the results of a collagen production test of fibroblasts of Reference Example 5.

FIG. 7 is a graph showing the results of a collagen production test of fibroblasts of Reference Example 5.

FIG. 8 is a graph showing the results of a hyaluronic acid production test of fibroblasts of Reference Example 5.

FIG. 9 is photos showing the results of a migration ability test of epidermal cells of Reference Example 6.

FIG. 10 is a graph showing the results of a migration ability test of epidermal cells of Reference Example 6.

### DESCRIPTION OF EMBODIMENTS

Hereafter, embodiments of the present invention are described in detail. It should be noted that the embodiments described below exemplify apparatuses and methods for embodying the technical idea of the present invention, and this technical idea of the present invention does not limit combinations of component members and the like to the following embodiments. Various changes can be made to the technical idea of the present invention in claims.

A method for providing a cosmetic or a pharmaceutical according to one embodiment comprises obtaining a supernatant of a medium of pluripotent stem cells derived from cells donated by a subject and providing the cosmetic or the pharmaceutical comprising the supernatant of the medium to the subject.

A makeup method according to one embodiment comprises obtaining a supernatant of a medium of pluripotent stem cells derived from cells donated by a subject and applying or administering the cosmetic comprising the supernatant of the medium to the subject.

A treatment method according to one embodiment comprises obtaining a supernatant of a medium of pluripotent stem cells derived from cells donated by a subject and applying or administering the pharmaceutical comprising the supernatant of the medium to the subject.

A cosmetic or a pharmaceutical according to one embodiment comprises a supernatant of a medium of pluripotent stem cells derived from cells donated by a subject and is used to be provided to the subject.

Use according to one embodiment is use of a supernatant of a medium of pluripotent stem cells derived from cells donated by a subject to manufacture a cosmetic or a pharmaceutical to be provided to the subject.

The subject is a human or non-human animal. The subject who donates cells and the subject to whom the cosmetic or the pharmaceutical is provided are identical. The cosmetic may be an autologous cosmetic. The pharmaceutical may be an autologous pharmaceutical.

Examples of pluripotent stem cells include Induced pluripotent stem cells (iPS cells) and embryonic stem cells.

The medium may be a medium of pluripotent stem cells cultured and maintained in an undifferentiated state. The medium may be a medium of pluripotent stem cells grown in adherent culture (one-dimensional culture). The medium may be a medium of pluripotent stem cells grown in suspension culture (two-dimensional culture). The medium may be a medium of pluripotent stem cells grown in three-dimensional stirring culture. The medium may be a medium of pluripotent stem cells grown in three-dimensional culture without stirring. The medium may be a gel medium.

Cells donated by a subject are not particularly limited. Examples of the cells donated by a subject include blood cells, fibroblasts, pulp stem cells, keratinocytes, oral cavity epithelial cells, and somatic stem precursor cells.

Examples of blood cells include mononuclear cell (Monocyte), nucleated cells, such as neutrophil, eosinophil, basophil, and lymphocyte, and do not include erythrocyte, granulocyte, or platelet. Blood cells may be, for example, vascular endothelial precursor cells, blood stem/precursor cells, T cells, or B cells. Examples of T cells include αβ T cells.

Methods for inducing iPS cells from cells donated by a subject are not particularly limited. For example, iPS cells are induced by introducing a reprogramming factor into cells donated by a subject. Examples of the reprogramming factor include Oct3/4, Sox2, Klf4, c-Myc, LIN28A, LIN28B, GLIS1, FOXH1, p53-dominant negative, p53-P275S, L-MYC, NANOG, DPPA2, DPPA4, DPPA5, ZIC3, BCL-2, E-RAS, TPT1, SALL2, NAC1, DAX1, TERT, ZNF206, FOXD3, REX1, UTF1, KLF2, KLF5, ESRRB, miR-291-3p, miR-294, miR-295, NR5A1, NR5A2, TBX3, MBD3sh, TH2A, and TH2B. The reprogramming factor may be a DNA, an RNA, and a protein.

Methods for introducing a reprogramming factor into cells donated by a subject are not particularly limited. Examples of the method for introducing a reprogramming factor into cells include electroporation, lipofection, and transfection using a vector. Examples of the vector include episomal vectors and Sendai virus (Sev).

The reprogramming factor may be introduced into cells using a chimeric virus having a virus-derived genomic RNA carrying a reprogramming factor RNA and an envelope enclosing the genomic RNA which is derived from a virus other than that of the genomic RNA.

The genomic RNA of a chimeric virus may be derived from paramyxovirus. The genomic RNA of a chimeric virus may be derived from Sendai virus. Sendai virus is a virus belonging to Mononegavirales paramyxovirus having RNA as a genome thereof. Wild type Sendai virus has an RNA genome and an envelope comprising a lipid bilayer membrane including RNA therein.

The genomic RNA of a chimeric virus may be a stealth RNA vector.

Among the Sendai virus genes including nucleocapsid protein (NP) gene, phosphorylation protein (P) gene/C protein (C) gene, matrix protein (M) gene, membrane fusion protein (F) gene, hemagglutinin-neuraminidase (HN) gene, and large protein (L) gene, the genomic RNA of a chimeric virus may have Sendai virus genes having mutations which cause deficiency in all functions of the M gene, the F gene, and the HN gene and can express the the L gene continuously, and a reprogramming factor RNA.

The envelope of a chimeric virus may be derived from measles virus.

A human ES/iPS medium, for example, such as TeSR2 (STEMCELL Technologies), can be used as a medium for stem cells to culture iPS cells. However, the medium for stem cells is not limited to this example, and various mediums for stem cells can be used. Example of the medium that can be used include Primate ES Cell Medium, Reprostem, ReproFF, ReproFF2, ReproXF (Reprocell), mTeSR1, TeSRE8, ReproTeSR (STEMCELL Technologies), PluriSTEM (registered trademark) Human ES/iPS Medium (Merck), NutriStem (registered trademark) XF/FF Culture Medium for Human iPS and ES Cells, Pluriton reprogramming medium (Stemgent), PluriSTEM (registered trademark), Stemfit AK02N, Stemfit AK03 (Ajinomoto), ESC-Sure (registered trademark) serum and feeder free medium for hESC/iPS (Applied StemCell), L7 (registered trademark) hPSC Culture System (LONZA), and Primate ES Cell Medium (ReproCELL).

Alternatively, the medium for stem cells can be Dulbecco's modified Eagle medium/nutrient mixture F-12 Ham (DMEM/F12) containing serum replacement, L-glutamine, nonessential amino acid solution, 2-mercaptoethanol, and penicillin/streptomycin. The medium for stem cells may contain a growth factor, such as basic fibroblast growth factor (bFGF).

When iPS cells are grown in suspension culture, a gel medium may be used, or a liquid medium may be used. The gel medium is prepared by, for example, adding a gellan gum, such as a deacylated gellan gum, to the medium for stem cells at a final concentration of 0.5% by weight to 0.001% by weight, 0.1% weight to 0.005% by weight, or 0.05% by weight to 0.01% by weight. It should be noted that the gellan gum includes a deacylated gellan gum in the present disclosure.

The gel medium may contain at least one type of polymer compound selected from the group consisting of hyaluronic acid, Rhamsan gum, diutan gum, xanthan gum, carrageenan, fucoidan, pectin, pectic acid, pectinic acid, heparan sulfate, heparin, heparitin sulfate, kerato sulfuric acid, chondroitin sulfate, dermatan sulfate, rhamnan sulfate, and salts thereof. Further, the gel medium may contain methylcellulose and lipids such as lysophosphatidic acid and sphingosine-1-phosphoric acid. Agglutination between cells can be controlled by adding these substances.

The gel medium may contain at least one temperature-sensitive gel selected from poly(glycerol monomethacrylate) (PGMA), poly(2-hydroxypropyl methacrylate) (PHPMA), Poly (N-isopropylacrylamide) (PNIPAM), amine terminated, carboxylic acid terminated, maleimide terminated, N-hydroxysuccinimide (NHS) ester terminated, triethoxysilane terminated, Poly (N-isopropylacrylamide-co-acrylamide), Poly (N-isopropylacrylamide-co-acrylic acid), Poly (N-isopropylacrylamide-co-butylacrylate), Poly (N-isopropylacrylamide-co-methacrylic acid), poly(N-isopropylacrylamide-co-methacrylic acid-co-octadecyl acrylate), and N-isopropylacrylamide.

A ROCK inhibitor may be added to the gel medium, for example, at a final concentration of 1000 μmol/L or higher and 0.1 μmol/L or lower, 100 μmol/L or higher and 1 μmol/L or lower, or 5 μmol/L or higher and 20 μmol/L or lower. Colony formation by stem cells is promoted by adding a ROCK inhibitor to a gel medium.

The gel medium does not have to contain, for example, a growth factor, such as bFGF. Or, the gel medium may contain a growth factor, such as bFGF, at a low concentration of 400 μg/L or lower, 100 μg/L or lower, 40 μg/L or lower, or 10 μg/L or lower.

Further, the gel medium may not contain TGF-β or contain TGF-β at a low concentration of 600 ng/L or lower, 300 ng/L or lower, or 100 ng/L or lower.

For example, iPS cells may be divided into single cells before they are grown in suspension culture, and iPS cells divided into single cells may be placed in the gel medium. The gel medium may be stirred or not stirred. Single cells grow while maintaining clonality and an undifferentiated state thereof, and they form colonies in the gel medium. Whether iPS cells maintain an undifferentiated state can be confirmed by examining whether cells are expressing an undifferentiation marker.

When iPS cells are cultured and maintained, the temperature is, for example, 37° C. When iPS cells are cultured and maintained, the concentration of carbon dioxide is, for example, 5%. The time period for which iPS cells are cultured and maintained is, for example, one or more days and 90 or less days, two or more days and 60 or less days, five or more days and 30 or less days, or seven or more days and 21 or less days. iPS cells may be removed from the supernatant of the medium in which iPS cells are cultured and maintained in an undifferentiated state by filtration, centrifugation, or the like.

iPS cells may be cultured and maintained to form clumps of iPS cells. For example, 50 to 600 clumps of iPS cells per milliliter of a medium are formed, but the number of clumps of iPS cells formed is not particularly limited. iPS cells in clumps may be cultured with a density of 50 to 600 clumps/mL or more for two or more days. When iPS cells are grown in suspension culture on a low attachment dish, some cell clumps may adhere to the low attachment dish lightly. When some cell clumps adhere to the low attachment dish lightly, cell clumps are hardly removed together with the medium being removed during medium exchange.

iPS cells may be grown in adherent culture or suspension culture in a bioreactor.

The supernatant of the medium of pluripotent stem cells derived from a subject may be examined for the effect on somatic cells derived from the subject. Subject-derived somatic cells may be skin cells. Somatic cells may be induced from subject-derived stem cells. For example, it may be examined whether the supernatant of the medium of pluripotent stem cells has at least one effect selected from the group consisting of the effect of preventing formation of and ameliorating skin blemishes, wrinkles, and sagging, the effect of promoting production of collagen, the effect of promoting production of hyaluronic acid, the effect of promoting dermal/epidermal cell growth, the effect of promoting production of fibroblast growth factor (FGF) family, the effect of promoting production of vascular endothelial growth factor (VEGF), the cytoprotective effect of protecting cells from stresses, the cell survival rate improving effect of improving the survival rate of cells suffering stresses, and the wound treatment effect on subject-derived somatic cells.

The pharmaceutical according to one embodiment may be a composition to be applied on the skin. The pharmaceutical according to one embodiment may be an agent for treating skin diseases. Examples of diseases that can be treated with a skin disease treating agent according to one embodiment include acne vulgaris, psoriasis vulgaris, keloid, seborrheic dermatitis, contact dermatitis, atopic dermatitis, atopic dry skin disease, dermatoporosis, photoelastic fibrosis, solar keratosis, eyelid ptosis, chloasma, senile lentigines, sudamina, freckles, delayed two-sided nevus of Ota, seborrheic keratosis, skin disease due to progeria, and simple herpes.

Examples of conditions that can be ameliorated or resolved with the cosmetic according to one embodiment include blemishes, freckles, wrinkles, sagging, friction, reduced suppleness of the skin, dullness, sensitive skin, and dry skin. Examples of the effect of the cosmetic according to one embodiment include conditioning the skin, fixing the skin's texture, keeping the skin healthy, preventing the skin from chapping, firming the skin, giving moisture to the skin, correcting and maintaining the water and oil content in the skin, maintaining softness of the skin, protecting the skin, preventing dryness of the skin, softening the skin, giving suppleness to the skin, making the skin radiant, smoothening the skin, making blemish less prominent, preventing wrinkles, and brightening the skin. The cosmetic or the pharmaceutical according to one embodiment may be infused into the skin. The region in the skin may be any of epidermis, dermis, and hypodermal tissue. Infusion may be performed by injection. Injection may be hydro-injection. Infusion may be performed using a microneedle. Infusion may be performed repeatedly. The cosmetic or the pharmaceutical according to one embodiment may be injected locally into the area of wrinkles or injected over the whole skin using a plurality of microneedles.

The pharmaceutical according to one embodiment may be an agent for treating wounds, a dermal/epidermal cell growth promoter, or an agent for promoting dermal/epidermal turnover. The pharmaceutical or the cosmetic according to one embodiment may be an agent for promoting production of collagen, an agent for promoting production of hyaluronic acid, an agent for promoting production of fibroblast growth factor (FGF) family, or an agent for promoting production of vascular endothelial growth factor (VEGF).

Examples of wounds that can be treated with a wound treating agent according to one embodiment include burn, abraded wound, laceration, bruise, suture wound, bedsore, and apellous wound.

The pharmaceutical or the cosmetic according to one embodiment may be a cytoprotective agent for protecting cells from stresses which comprises the supernatant of the medium of iPS cells cultured and maintained in an undifferentiated state. Further, the pharmaceutical or the cosmetic according to one embodiment may be, for example, a cell survival rate improving agent comprising the supernatant of the medium of iPS cells cultured and maintained in an undifferentiated state which stabilizes cells suffering stresses and improves the survival rate. Cells suffering stresses are, for example, fibroblasts, dermal cells, and epidermal cells, but are not limited to these cells and can be any cells. The pharmaceutical or the cosmetic according to one embodiment may be a biomaterial protecting agent comprising the supernatant of the medium of iPS cells cultured and maintained in an undifferentiated state which protects at least one biomaterial selected from the group consisting of nucleic acids, proteins, protein composites, lipoproteins, ribosomes, and biomembranes from stresses. Here, biomembranes include cell membranes.

The pharmaceutical and the cosmetic according to one embodiment comprise an effective amount of the supernatant of the medium of stem cells cultured and maintained in an undifferentiated state. Here, the effective amount is defined as the amount that can exhibit an effect as the pharmaceutical or the cosmetic. The effective amount is suitably selected depending on the patient's age, target disease, presence of other success ingredients, and the amounts of other compositions.

The pharmaceutical and the cosmetic according to one embodiment may contain pharmaceutically acceptable carriers, excipients, disintegrating agents, buffers, emulsifiers, suspensions, soothing agents, stabilizers, preserving agents, preservatives, physiological saline, and like. Examples of excipients include lactose, starch, sorbitol, D-mannitol, and sucrose. Examples of disintegrating agents include carboxymethylcellulose and calcium carbonate. Examples of buffers include phosphates, citrates, and acetates. Examples of emulsifiers include gum arabic, sodium alginate, and tragacanth.

Examples of suspensions include glycerin monostearate, aluminium monostearate, methylcellulose, carboxymethylcellulose, hydroxymethylcellulose, and sodium lauryl sulfate. Examples of soothing agents include benzyl alcohol, chlorobutanol, and sorbitol. Examples of stabilizers include propylene glycol and ascorbic acid. Examples of preserving agents include phenol, benzalkonium chloride, benzyl alcohol, chlorobutanol, and methyl paraben. Examples of preservatives include benzalkonium chloride, para-hydroxybenzoic acid, and chlorobutanol.

The pharmaceutical and the cosmetic according to one embodiment can contain water, alcohols, surfactants (e.g., cation, anion, nonion, and ampholytic surfactant), moisturizing agents (e.g., glycerin, 1,3-butylene glycol, propylene glycol, propane diol, pentane diol, polyquaternium, amino acids, urea, pyrrolidone carboxylate, nucleic acids, monosaccharides, and oligosaccharides and derivatives thereof), thickeners (e.g., polysaccharides, polyacrylates, carboxy vinyl polymers, polyvinylpyrrolidone, polyvinyl alcohol, chitin, chitosan, algic acid, carrageenan, xanthan gum, and methylcellulose and derivatives thereof), wax, Vaseline, hydrocarbon saturated fatty acid, unsaturated fatty acid, and silicon oil and derivatives thereof, triglycerides, such as tri (caprylic/capric acid)glyceryl and trioctanoate glyceryl, ester oils such as isopropyl stearate, natural oils and fats (e.g., olive oil, camellia oil, avocado oil, almond oil, cacao butter, evening primrose oil, grape seed oil, macadamia nut oil, eucalyptus oil, rosehip oil, squalane, orange roughy oil, lanolin, and ceramide), preservatives (e.g., oxy benzoic acid derivatives, dehydroacetates, photosensitizers, sorbic acid, and phenoxy ethanol and derivatives thereof), sterilizers (e.g., sulfur, trichlorocarbanilide, salicylic acid, zinc pyrithione, and hinokitiol and derivatives thereof), ultraviolet ray absorbing agents (e.g., p-aminobenzoic acid and methoxycinnamic acid and derivatives thereof), anti-inflammation agents (e.g., allantoin, bisabolol, ε-aminocapronic acid, acetyl farnesyl cysteine, and glycyrrhizinic acid and derivatives thereof), anti-oxidizing agents (e.g., tocopherol, BHA, BHT, and astaxanthin and derivatives thereof), chelating agents (e.g., edetic acid and hydroxyethane diphosphonic acid and derivatives thereof), animal and plant extracts (e.g., Angelica keiskei, aloe, rose fruit, scutellaria root, cork tree bark, seaweeds, quince, chamomile flower, licorice, kiwi fruit, cucumber, mulberry, Japanese white birch, Japanese angelica root, garlic, peony, hop, horse chestnut, lavender, rosemary, eucalyptus, milk, various peptides, placenta, royal jelly, euglena extract, hydrolyzed euglena extract, and euglena oil and products of purification or fermentation of these ingredients), pH modifiers (e.g., mineral acids, mineral acid salts, organic acids, and organic acid salts and derivatives thereof), vitamins (vitamins A family, vitamins B family, vitamin C, vitamins D family, ubiquinone, and nicotinic acid amide and derivatives thereof), yeast, koji mold, and ferment filtrate of lactic bacteria, galactomyces ferment filtrate, skin-lightening agents (e.g., tranexamic acid, cetyl tranexamate hydrochloride, 4-n-butyl resorcinol, arbutin, kojic acid, ellagic acid, licorice flavonoid, niacin amide, and vitamin C derivatives), ceramide/ceramide derivatives, anti-wrinkle agents (e.g., retinol and retinal and derivatives thereof, nicotinic acid amide, oligopeptides, and the like and derivatives thereof, and natural and synthetic components having an effect of inhibiting neutrophil elastase and an effect of inhibiting MMP-1 and MMP-2), titanium oxide, talc, mica, silica, zinc oxide, iron oxide, silicon, powders obtained by processing these ingredients, and others as long as the objective of the pharmaceutical and the cosmetic according to one embodiment is achieved.

It should be noted that components that can be added to the pharmaceutical or the cosmetic according to one embodiment are not limited to the above-mentioned substances, and any component that can be used in pharmaceuticals or cosmetics can be selected freely. When the pharmaceutical or the cosmetic according to one embodiment is used as a poultice, a solvent (e.g., kaolin and bentonite) and a gelling agent (e.g., polyacrylic acid salts and polyvinyl alcohol) can be mixed in addition to the above-mentioned components as long as the objective is achieved. When the pharmaceutical or the cosmetic according to one embodiment is used as a bath additive, sulfuric acid salts, hydrogen carbonates, boric acid salts, dyes, and moisturizing agents may be mixed suitably as long as the objective is achieved, so that the bath additive can be prepared as a powder or a solution.

The pharmaceutical and the cosmetic according to one embodiment can be manufactured by a method known and usually used in the technical field.

The pharmaceutical and the cosmetic according to one embodiment exhibit a large treatment effect and a large cosmetic effect because a subject who donates cells and a subject to whom the cosmetic or the pharmaceutical is provided are identical.

### Example 1: Preparation of iPS Cells

Human skin fibroblasts were collected from each of a first individual and a second individual. OCT3/4 RNA, SOX2 RNA, KLF4 RNA, and c-MYC RNA were introduced into human skin fibroblasts using an episomal vector to induce iPS cells.

### Example 2: Preparation of Supernatant of Medium in which Stem Cells were Cultured and Maintained

DMEM/F12 (10565-018, Invitrogen) and serum replacement (KnockOut Serum Replacement [KSR], registered trademark, 10828028, Invitrogen) were added to 5 mL of L-glutamine (25030-081, Invitrogen), 5 mL of a nonessential amino acid solution (11140-050, Invitrogen), 1 mL of 2-mercaptoethanol (21985-023, Invitrogen), and 2.5 mL of penicillin/streptomycin (15140-122, Invitrogen) to prepare a medium having a total volume of 500 mL. In an amount of 0.2 mL of 10 μg/mL basic fibroblast growth factor (bFGF, R & D) was added to this medium to prepare a medium for stem cells. The concentration of bFGF in the medium for stem cells was 4 ng/mL.

Using the medium for stem cells prepared as described above, iPS cells were cultured on a plate for adherent culture which was coated with laminin 511 (Nippi). The iPS cells were passaged every week. During the passage, the iPS cells were treated with a solution for dissociating ES cells (TrypLE Select, registered trademark, Thermo Fisher Scientific Inc.).

The iPS cells cultured and maintained as described above were detached from the plate for adherent culture using a solution for dissociating ES cells (TrypLE Select, registered trademark, Thermo Fisher Scientific Inc.). The detached iPS cells were seeded on a dish coated with laminin 511 (Nippi). Then, the iPS cells were cultured for one week using a Puel medium (I Peace Inc.) containing 10 μmol/L ROCK inhibitor to increase the density of the iPS cells. The medium was exchanged every day.

Then, the medium was replaced with a medium for stem cells to culture and maintain the iPS cells, and the supernatant of the medium for stem cells was recovered two days later. The recovered supernatant of the medium for stem cells was centrifuged at 1500 rpm for five minutes, the supernatant of the medium was recovered again and then centrifuged at 3000 rpm for three minutes, and the supernatant of the centrifuged medium for stem cells was filtrated through a 0.22 μm filter. The filtrated supernatant of the medium for stem cells was used as a supernatant solution of Example 2.

It was confirmed that undifferentiation markers LIN28, OCT3/4, and TRA 1-60 were positive in the cultured and maintained iPS cells.

### Example 3: Preparation of Supernatant of Medium in which Stem Cells were Cultured and Maintained

Human iPS cells were cultured and maintained in the same manner as in Example 2. Then, the human iPS cells were detached from the plate for adherent culture in the same manner as in Example 2 and divided into single cells. Then, the human iPS cells were seeded at 3.6×10⁶/10 mL on a medium for stem cells which was gelatinized by adding gellan gum and 10 μmol/L ROCK inhibitor (Selleck), and the human iPS cells were cultured and maintained in suspension for 21 days to form clumps of 100 or more iPS cells per milliliter. During this process, the gelatinized medium for stem cells was added to an incubator every other day.

Then, the gelatinized medium for stem cells in which the human iPS cells were suspended was filtrated through a mesh filter to remove cell clumps. Further, the gelatinized medium for stem cells filtrated through the filter was centrifuged at 1500 rpm for five minutes to precipitate cells and gel, the supernatant of the centrifuged medium for stem cells was recovered again and then centrifuged at 3000 rpm for three minutes, and the supernatant of the centrifuged medium for stem cells was filtrated through a 0.22 μm filter. The filtrated supernatant of the medium for stem cells was used as a supernatant solution of Example 3.

It was confirmed that undifferentiation markers LIN28, OCT3/4, and TRA 1-60 were positive in the cultured and maintained iPS cells.

### Example 4: Preparation of Supernatant of Medium in which Stem Cells were Cultured and Maintained

Human iPS cells were cultured and maintained in the same manner as in Example 2. Then, the human iPS cells were detached from the plate for adherent culture in the same manner as in Example 2 and divided into single cells. The iPS cells were seeded at 3.6×10⁶/10 mL on a medium for stem cells on a low attachment dish and cultured and maintained in suspension for six days to form clumps of 100 or more iPS cells per milliliter. Some cell clumps were attached lightly to the low attachment dish. During this process, 10 mL of the medium for stem cells was added every other day.

Then, the medium for stem cells in which the human iPS cells were suspended was filtrated through a mesh filter to remove cell clumps. Further, the filtrated medium for stem cells was centrifuged at 1500 rpm for five minutes to precipitate the cells, the supernatant of the centrifuged medium for stem cells was recovered again and then centrifuged at 3000 rpm for three minutes, and the supernatant of the centrifuged medium for stem cells was filtrated through a 0.22 μm filter. The filtrated supernatant of the medium for stem cells was used as a supernatant solution of Example 4.

It was confirmed that undifferentiation markers LIN28, OCT3/4, and TRA 1-60 were positive in the cultured and maintained iPS cells.

### Example 5: Preparation of Supernatant of Medium in which Stem Cells were Cultured and Maintained

Human iPS cells were cultured and maintained in the same manner as in Example 2. Then, the human iPS cells were detached from the plate for adherent culture in the same manner as in Example 2 and divided into single cells. The iPS cells were seeded at 3.6×10⁶/10 mL on a medium for stem cells in a bioreactor and cultured and maintained in suspension for six days to form clumps of 100 or more iPS cells per milliliter. During this process, 10 mL of the medium for stem cells was added every other day.

Then, the medium for stem cells in which the human iPS cells were suspended was filtrated through a mesh filter to remove cell clumps. Further, the filtrated medium for stem cells was centrifuged at 1500 rpm for five minutes to precipitate the cells, the supernatant of the centrifuged medium for stem cells was recovered again and then centrifuged at 3000 rpm for three minutes, and the supernatant of the centrifuged medium for stem cells was filtrated through a 0.22 μm filter. The filtrated supernatant of the medium for stem cells was used as a supernatant solution of Example 5.

It was confirmed that undifferentiation markers LIN28, OCT3/4, and TRA 1-60 were positive in the cultured and maintained iPS cells.

### Example 6: Type I Collagen Production Test Using Fibroblasts

A DMEM medium containing 10% FBS and 1% penicillin-streptomycin was prepared as a growth medium A. Then, adult-derived normal human fibroblasts (KF-4109, Strain No. 01035, Kurabo Industries Ltd.) were suspended in the growth medium A at a concentration of 5×10³ cells/0.1 mL/well, seeded on a 96-well plate, and cultured in a CO₂ incubator (5% CO₂, 37° C.) for one day.

A DMEM medium containing 1% FBS and 1% penicillin-streptomycin was prepared as a test medium A. Then, each of the supernatant solutions of Examples 2 to 5 and the test medium A were mixed in a volume ratio of 50.00:50.00 to obtain mediums A respectively containing the supernatants of Examples 2 to 5. The growth mediums A in some wells were replaced with the mediums A containing the supernatants of Examples 2 to 5, respectively, and the growth medium A in other wells was replaced with the test medium A not containing a supernatant solution.

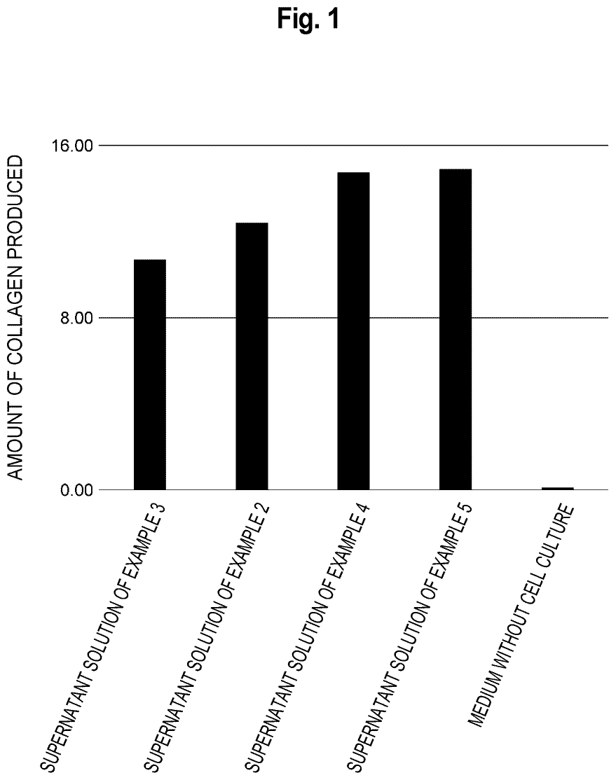

The fibroblasts were cultured in the replaced mediums for three days, and the supernatants of the mediums were recovered and stored at −80° C. Then, the supernatants of the mediums were thawed, and the concentration of type I collagen in the supernatants of the mediums was measured using a human collagen type I ELISA kit (Cat. No. EC1-E105). The results are shown in FIG. 1.

### Example 7: Type I Collagen Production Test Using Fibroblasts

A culture supernatant solution of iPS cells derived from the first individual of Example 2 and a culture supernatant solution of iPS cells derived from the second individual of Example 2 were prepared. Further, fibroblasts derived from the first individual and fibroblasts derived from the second individual were prepared.

The fibroblasts derived from the first individual were prepared by the following procedure. iPS cells derived from the first individual were prepared by the same method as in Example 1. The iPS cells were detached from the dish and seeded at 3.6×10⁶/10 mL on a low attachment plate. Then, the cells were cultured in a human ES medium not containing bFGF for nine days. Cell clumps were seeded on a gelatin-coated dish on Day 9, and the cells were cultured in a human ES medium not containing bFGF for further nine days. Then, the cells were detached from the dish using trypsin, seeded on a gelatin-coated dish, and cultured in a 10% FBS-containing DMEM. Then, the cells were passaged every seven days three times to obtain fibroblasts. The fibroblasts derived from the second individual were prepared in the same manner as the fibroblasts derived from the first individual.

By the same method as in Example 5, the fibroblasts were cultured under the following conditions: a combination of the culture supernatant solution of the iPS cells derived from the first individual and the fibroblasts derived from the first individual, a combination of the culture supernatant solution of the iPS cells derived from the first individual and the fibroblasts derived from the second individual, a combination of the culture supernatant solution of the iPS cells derived from the second individual and the fibroblasts derived from the first individual, and a combination of the culture supernatant solution of the iPS cells derived from the second individual and the fibroblasts derived from the second individual. The amount of type I collagen produced was investigated. The amount of type I collagen produced was detected using ELISA Kit for Collagen Type I (COL1) (Cloud-Clone Corp.).

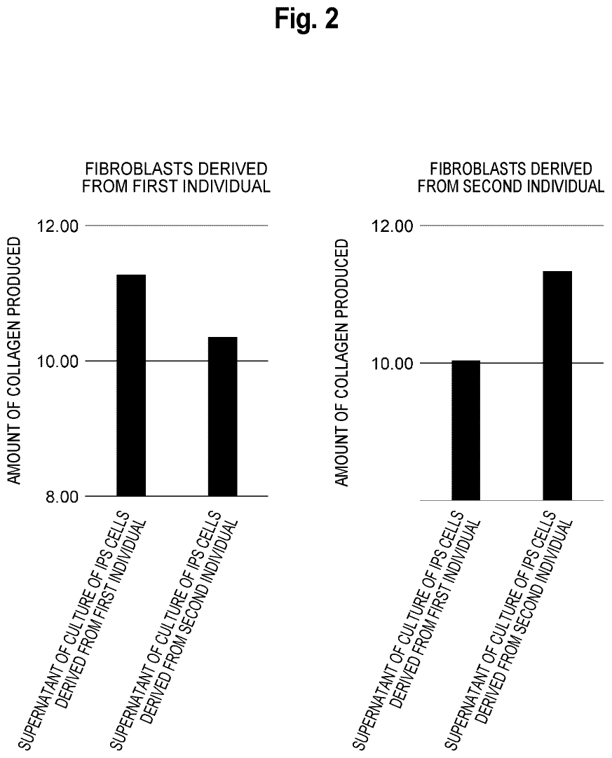

From the results, it was confirmed that, when iPS cells and fibroblasts were derived from the same individual, the amount of collagen produced by fibroblasts increased, as shown in FIG. 2.

### Example 8: Treatment by Hydro-Injection

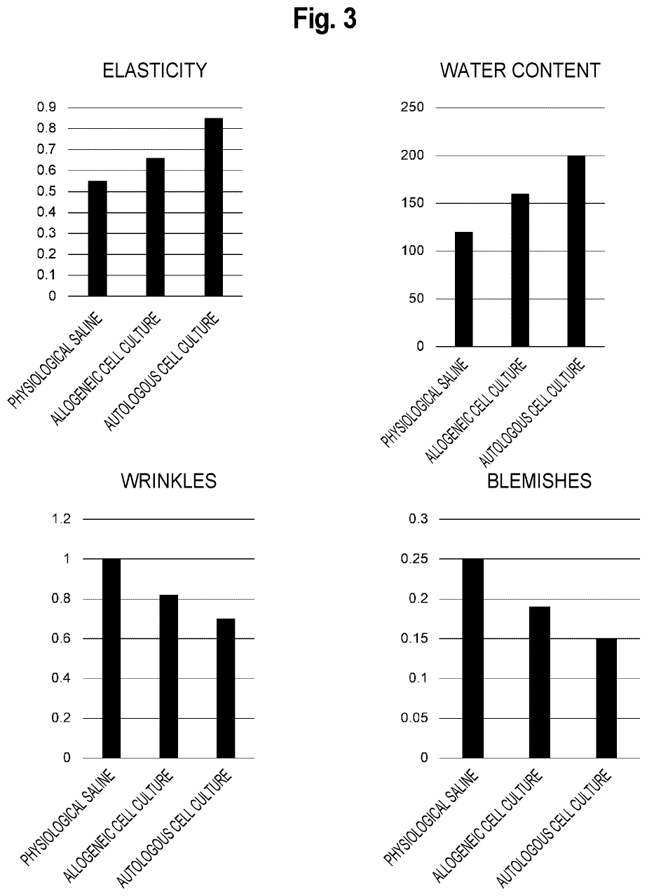

Into the dermis of the face of a female human subject, the following were subcutaneously injected using a microneedle: physiological saline; a solution obtained by diluting a culture supernatant solution of iPS cells derived from the subject with physiological saline to 10%, the supernatant solution prepared by the same method as in Example 2; and a solution obtained by diluting a culture supernatant solution of iPS cells derived from a human who was not the subject with physiological saline to 10%, the supernatant solution prepared by the same method as in Example 2. Then, the subject's skin elasticity, water content, wrinkles, and blemishes were analyzed. The skin elasticity was measured using Cutometer DUAL MPA580 (Integral Corporation). The water content of the skin was measured using SKICON-200EX-USB (Yayoi Corporation). Skin wrinkles and blemishes were measured using VISIA Evolution (registered trademark, Integral Corporation). As shown in FIG. 3, the results showed that the culture supernatant solution of the subject-derived iPS cells showed the best amelioration in terms of all the subject's skin elasticity, water content, wrinkles, and blemishes. It should be noted that the vertical axis in FIG. 3 represents the relative value in evaluation.

### Reference Example 1: Preparation of Supernatant of Medium in which Stem Cells were Cultured and Maintained

DMEM/F12 (10565-018, Invitrogen) and serum replacement (KnockOut Serum Replacement [KSR], registered trademark, 10828028, Invitrogen) were added to 5 mL of L-glutamine (25030-081, Invitrogen), 5 mL of a nonessential amino acid solution (11140-050, Invitrogen), 1 mL of 2-mercaptoethanol (21985-023, Invitrogen), and 2.5 mL of penicillin/streptomycin (15140-122, Invitrogen) to prepare a medium having a total volume of 500 mL. 0.2 mL of 10 μg/mL basic fibroblast growth factor (bFGF, R & D Systems, Inc.) was added to this medium to prepare a medium for stem cells. The concentration of bFGF in the medium for stem cells was 4 ng/ml.

Using the medium for stem cells prepared as described above, human iPS cells were adhered and cultured and maintained on feeder cells on a plate for adherent culture. The human iPS cells were passaged every week. During the passage, the human iPS cells were treated with a cell detachment solution containing 0.25% trypsin, 0.1 mg/mL collagenase IV, 1 mmol/L CaCl₂), and 20% KSR.

The human iPS cells cultured and maintained as described above were detached from the plate for adherent culture using a solution for dissociating ES cells (TrypLE Select, registered trademark, Thermo Fisher Scientific Inc.). The detached human iPS cells were seeded on a dish coated with laminin (Nippi). Then, the human iPS cells were cultured for one week using TeSR2 medium (STEMCELL Technologies) containing 10 μmol/L ROCK inhibitor. The medium was exchanged every day.

Then, the medium was replaced with a medium for stem cells, and the supernatant of the medium for stem cells was recovered two days later. The recovered supernatant of the medium for stem cells was centrifuged at 1500 rpm for five minutes, the supernatant of the medium was recovered again and then centrifuged at 3000 rpm for three minutes, and the supernatant of the centrifuged medium for stem cells was filtrated through a 0.22 μm filter. The filtrated supernatant of the medium for stem cells was used as a supernatant solution of Reference Example 1.

It was confirmed that undifferentiation markers NANOG, OCT3/4, and TRA 1-60 were positive in the cultured and maintained iPS cells.

### Reference Example 2: Preparation of Supernatant of Medium in which Stem Cells were Cultured and Maintained

Human iPS cells were cultured and maintained in the same manner as in Reference Example 1. Then, the human iPS cells were detached from the plate for adherent culture in the same manner as in Reference Example 1 and divided into single cells. Then, the human iPS cells were seeded on a medium for stem cells which was gelatinized by adding gellan gum and 10 μmol/L ROCK inhibitor (Selleck), and the human iPS cells were cultured and maintained in suspension for 21 days. During this process, the gelatinized medium for stem cells was replenished in an incubator every other day.

Then, the gelatinized medium for stem cells in which the human iPS cells were suspended was filtrated through a mesh filter to remove cell clumps. Further, the filtrated gelatinized medium for stem cells was centrifuged at 1500 rpm for five minutes to precipitate the cells and gel, the supernatant of the centrifuged medium for stem cells was recovered again and then centrifuged at 3000 rpm for three minutes, and the supernatant of the centrifuged medium for stem cells was filtrated through a 0.22 μm filter. The filtrated supernatant of the medium for stem cells was used as a supernatant solution of Reference Example 2.

It was confirmed that undifferentiation markers NANOG, OCT3/4, and TRA 1-60 were positive in the cultured and maintained iPS cells.

### Reference Example 3: Preparation of Supernatant of Medium in which Stem Cells were Cultured and Maintained

Human iPS cells were cultured and maintained in the same manner as in Reference Example 1. Then, the human iPS cells were detached from the plate for adherent culture in the same manner as in Reference Example 1 and divided into single cells. Then, the human iPS cells were seeded on a medium for stem cells which was gelatinized by adding gellan gum and 100 μmol/L ROCK inhibitor (Selleck), and the human iPS cells were cultured and maintained in suspension for 14 days. During this process, the gelatinized medium for stem cells was replenished in an incubator every other day.

Then, the gelatinized medium for stem cells in which the human iPS cells were suspended was filtrated through a mesh filter to remove cell clumps. Further, the filtrated gelatinized medium for stem cells was centrifuged at 1500 rpm for five minutes to precipitate the cells and gel, the supernatant of the centrifuged medium for stem cells was recovered again and then centrifuged at 3000 rpm for three minutes, and the supernatant of the centrifuged medium for stem cells was filtrated through a 0.22 μm filter. The filtrated supernatant of the medium for stem cells was used as a supernatant solution of Reference Example 3.

It was confirmed that undifferentiation markers NANOG, OCT3/4, and TRA 1-60 were positive in the cultured and maintained iPS cells.

### Reference Comparative Example: Preparation of Supernatant of Medium in which Differentiated Cells were Cultured and Maintained

Human iPS cells were cultured according to an example described in Japanese Patent Laid-open No. 2016-128396. Specifically, using the same medium for stem cells as used in Reference Example 1, human iPS cells were adhered and cultured and maintained on feeder cells on the plate for adherent culture. The human iPS cells were passaged every week. During the passage, the human iPS cells were treated with a cell detachment solution containing 0.25% trypsin, 0.1 mg/ml collagenase IV, 1 mmol/L CaCl₂), and 20% KSR.

The human iPS cells cultured as described above were detached from the plate for adherent culture using a solution for dissociating ES cells (TrypLE Select, registered trademark, Thermo Fisher Scientific Inc.). The detached human iPS cells were grown for one week in suspension culture of human iPS cells which was not gelatinized and was placed in the plate for nonadherent culture. As a result, embryoids (EBs) were formed. The formed EBs were seeded on the plate for adherent culture and grown in a DMEM containing 10% FBS for one week (outgrowth).

Then, the cells were detached from the plate for adherent culture using a 0.05% trypsin-EDTA solution, and the cells divided into single cells were seeded on a plate for adherent culture. Then, the cells were cultured for one week using a DMEM containing 10% FBS as a medium.

After it was confirmed that the cells had reached 70% confluency to higher than 80% confluency, the medium was replaced with a serum-free medium (a DMEM not containing FBS), and the supernatant of the medium was recovered after cultured for two days. The medium supernatant was centrifuged at 1500 rpm for five minutes, the supernatant of the medium was recovered again and then centrifuged at 3000 rpm for three minutes, and the supernatant of the medium recovered again was used as a supernatant solution of Reference Comparative Example.

It was confirmed that undifferentiation markers NANOG, OCT3/4, and TRA 1-60 were negative in the cultured cells, indicating that cells had been differentiated.

### Reference Example 4: Fibroblast Proliferative Ability Test

A DMEM medium containing 10% FBS and 1% penicillin-streptomycin was prepared as a growth medium A. Then, adult-derived normal human fibroblasts (KF-4109, Strain No. 01035, Kurabo Industries Ltd.) were suspended in the growth medium A at a concentration of 5×10³ cells/0.1 mL/well, seeded on a 96-well plate, and cultured in a CO₂ incubator (5% CO₂, 37° C.) for one day.

A DMEM medium containing 1% FBS and 1% penicillin-streptomycin was prepared as a test medium A. Then, any of the supernatant solutions of Reference Examples 1 to 3 and Reference Comparative Example and the test medium A were mixed in a volume ratio of 10.00:90.00 to obtain mediums A respectively containing the supernatants of Reference Examples 1 to 3 and Reference Comparative Example at a concentration of 10.00 v/v %. The growth mediums A in some wells were replaced with the mediums A containing the supernatants of Reference Examples 1 to 3 and Reference Comparative Example, respectively.

As a negative control, the growth medium A in other wells was replaced with a DMEM medium not containing 1% FBS or 1% penicillin-streptomycin (a test medium A with no addition). Further, as a negative control, DMEM/F12 and the test medium A were mixed in a volume ratio of 10.00:90.00, and the growth medium A in some wells was replaced with the obtained diluted test medium A.

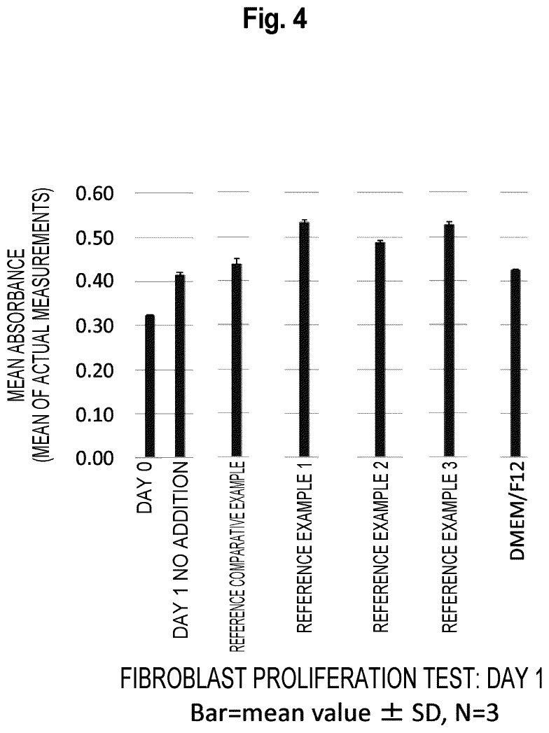

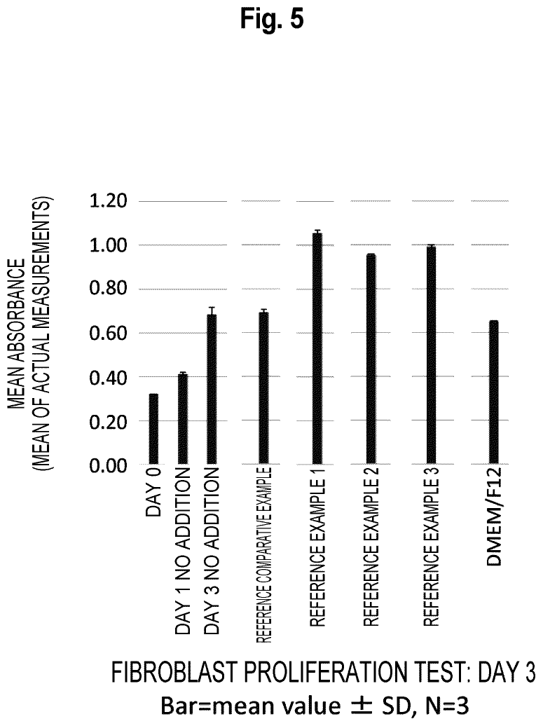

Fibroblasts were cultured in the replaced medium for one day and three days, and viable cells were counted by a WST-8 assay using a viable cell count reagent SF (Cat. No. 07553-15, Nacalai Tesque) and a plate reader (Varioskan MicroPlate Reader, Thermo Scientific). The results are shown in FIGS. 4 and 5. When the medium A containing any of the supernatants of Reference Examples 1 to 3 at a concentration of 10.00 v/v % was used, a superior growth of fibroblasts was confirmed as compared with the test medium A with no addition, the diluted test medium A, and the medium A containing the supernatant of Reference Comparative Example. Additionally, it was shown that the supernatant of the medium of stem cells was effective to increase the cell survival rate. When detached from the plate during a passage or divided into single cells, cells suffer physical stresses such as pressure and chemical stresses from the cell detachment agent. However, since the cells suffering these stresses grew in the mediums containing the supernatants of Reference Examples, it was indicated that the mediums containing the supernatants of Reference Examples reduced the stresses imposed on cells, protected cells from the stresses, and stabilized cells, thus improving the survival rate. These results indicated that the mediums containing the supernatants of Reference Examples protected nucleic acids, proteins, protein composites, lipoproteins, ribosomes, and biomembranes contained in cells. Here, biomembranes include cell membranes.

### Reference Example 5: Type I Collagen and Hyaluronic Acid Production Tests of Fibroblasts

Adult-derived normal human fibroblasts were cultured in the growth medium A for one day in the same manner as in Reference Example 4. Then, the growth mediums A in some wells were replaced with the mediums A containing the supernatants of Reference Examples 1 to 3 and Reference Comparative Example, respectively, in the same manner as in Reference Example 4, except that the concentration was 1.00 v/v %, 10.00 v/v %, or 100.0 v/v %.

As a positive control, the growth medium A in some wells was not replaced. As a negative control, the growth medium A in other wells was replaced with a test medium A with no addition. Further, as a negative control, the growth medium A in some wells was replaced with a diluted test medium A prepared in the same manner as in Reference Example 4.

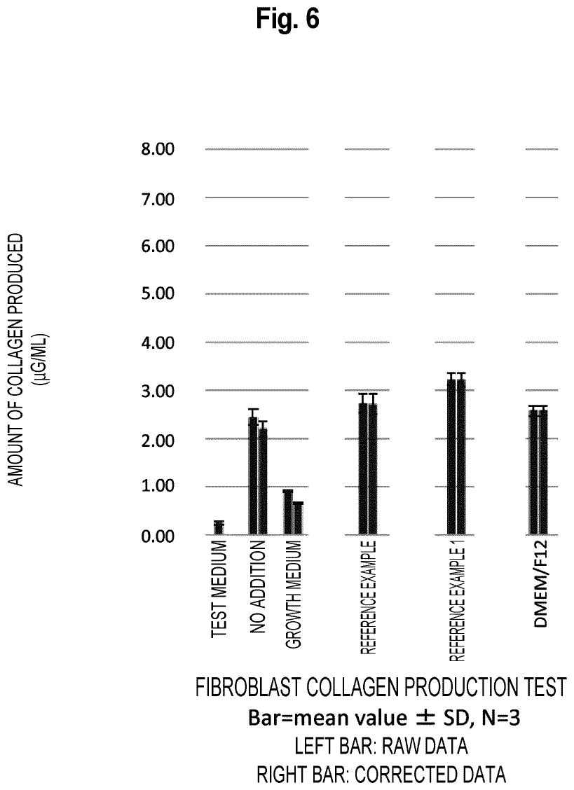

After the medium was exchanged, the fibroblasts were cultured for three days, and the supernatant of the medium was recovered and stored at −80° C. Then, the supernatant of the medium was thawed, and the concentration of type I collagen in the supernatant of the medium was measured using a human collagen type I ELISA kit (Cat. No. EC1-E105). Further, the concentration of hyaluronic acid in the supernatant of the medium was measured using a Hyaluronan DuoSet kit (Cat. No. DY3614, R & D Systems). The results are shown in FIGS. 6, 7, and 8.

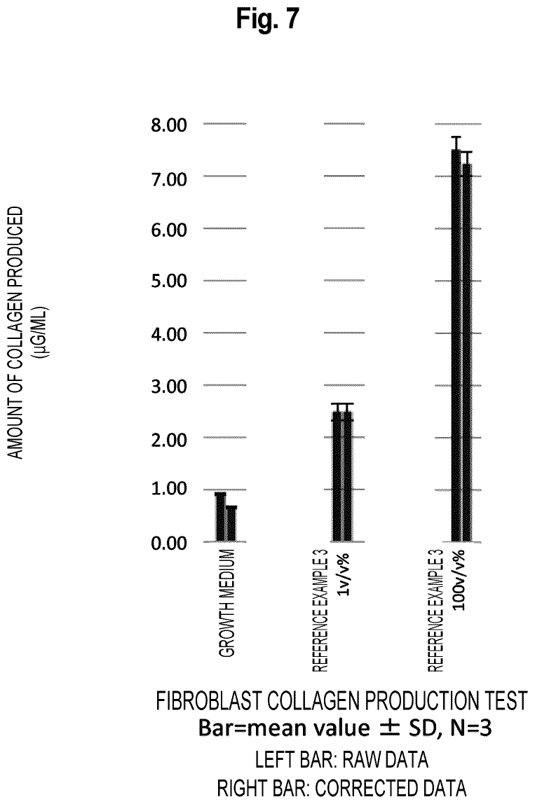

In FIGS. 6 and 7, the corrected bar on the right side represents data corrected by excluding the amount of collagen contained in the original medium before fibroblasts were cultured. The raw data bar on the left side represents data before correction. As shown in FIG. 6, when the medium A containing the supernatant of Reference Example 1 at a concentration of 1.0 v/v % was used, the increase in the amount of type I collagen produced was superior to those in the test medium A with no addition, the growth medium A, the diluted test medium A, and the medium A containing the supernatant of Reference Comparative Example. As shown in FIG. 7, when the medium A containing the supernatant of Reference Example 3 at a concentration of 1.0 v/v % or 100.0 v/v % was used, the amount of type I collagen produced increased markedly as compared with when the growth medium A was used. Therefore, it was indicated that the supernatant of the medium of stem cells was effective to promote the production of collagen and prevent formation of and ameliorate skin wrinkles and sagging.

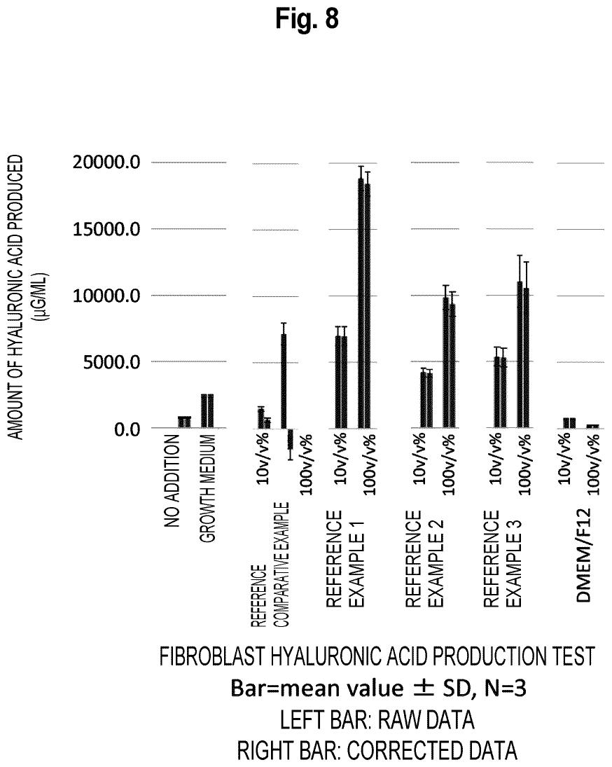

In FIG. 8, the corrected bar on the right side shows data corrected by excluding the amount of hyaluronic acid contained in the original medium before fibroblasts were cultured. The raw data bar on the left side represents data before correction. When the medium A containing any of the supernatants of Reference Examples 1 to 3 at a concentration of 10.0 v/v % or 100.0 v/v % was used, the amount of hyaluronic acid produced increased markedly as compared with the test medium A with no addition, the diluted test medium A, and the medium A containing the supernatant of Reference Comparative Example. Therefore, it was indicated that the supernatant of the medium of the stem cells was effective to promote the production of hyaluronic acid, prevent formation of wrinkles and sagging due to reduction of hyaluronic acid, and ameliorate wrinkles and sagging.

### Reference Example 6: Epidermal Cell Migration Test

As a growth medium B, 500 mL of epidermal cell medium (HuMedia-KG2, Kurabo Industries Ltd.) containing additives for growth (10 μg/mL insulin, 0.1 ng/ml hEGF, 0.67 μg/mL hydrocortisone, and 4 μL/mL bovine pituitary extraction [BPE]) and antibacterial agents (50 μg/mL gentamicin and 50 ng/mL amphotericin) was prepared.

Adult-derived normal human epidermal cells were treated with 10 μg/mL Mitomycin C (Cat. No. 20898-21, Nacalai Tesque) for two hours to arrest cell division. Then, the human epidermal cells were suspended in the growth medium B at a concentration of 4×10⁴ cells/0.1 mL/well, seeded on a collagen-coated plate of a kit for measuring the cell migration ability (Oris Cell Migration Assay, registered trademark), cultured in a CO₂ incubator (5% CO₂, 37° C.) for one day, and fixed on the outer edge of a stopper on the plate where the stopper was not blocked. Then, the stopper was removed from the plate.

As a test medium B, a medium was prepared by adding 500 mL of antibacterial agents (50 μg/mL gentamicin and 50 nm/mL amphotericin) to an epidermal cell medium. Then, any of the supernatant solutions of Reference Examples 1 to 3 and Reference Comparative Example and the test medium B were mixed in volume ratios of 10.0:90.0 and 1.0:99.0 to obtain mediums B respectively containing the supernatants of Reference Examples 1 to 3 and Reference Comparative Example. The growth mediums B on some plates were replaced with the mediums B containing the supernatants of Reference Examples 1 to 3 and Reference Comparative Example, respectively.

As a negative control, the growth medium B on some plates was replaced with an epidermal cell medium not containing growth additives (a test medium B with no addition). Additionally, as a negative control, the growth medium B on some plates was replaced with a diluted test medium B obtained by mixing DMEM/F12 and the test medium B in volume ratios of 10.0:90.0 and 1.0:99.0.

During wound cure, epidermal cells migrate toward the wound, and the wound shrinks. In this Reference Example, whether the epidermal cells migrated to a place blocked by a stopper was analyzed using a plate reader. Specifically, 23 hours after the medium was replaced, the epidermal cells were stained using a reagent for staining viable cells (Calcein AM, Cat. NO. 341-07901, DOJINDO), and fluorescence at a wavelength of 538 nm against excitation light at a wavelength of 485 nm was measured using a plate reader (Varioskan MicroPlate Reader, Thermo Scientific).

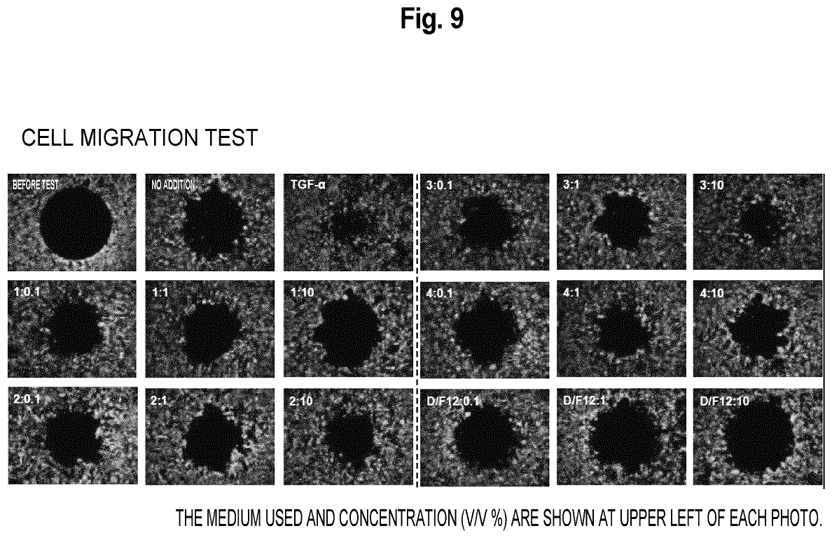

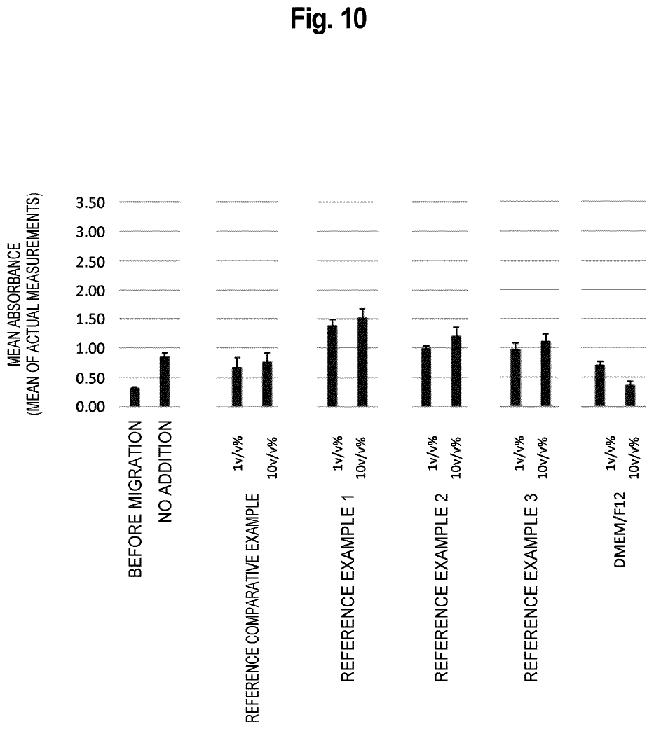

The results are shown in FIGS. 9 and 10. When the medium B containing any of supernatants of Reference Examples 1 to 3 at a concentration of 1.0 v/v % or 10.0 v/v % was used, the significant effect of promoting the migration ability of epidermal cells was exhibited as compared with test medium B with no addition, the diluted test medium B, and the medium B of the supernatant of Reference Comparative Example. Therefore, it was shown that the supernatant of the medium of stem cells was effective for wound cure. Further, it was indicated that, for example, the supernatant of the medium of stem cells was effective to prevent of formation of and ameliorate blemishes and uneven color of the skin surface layer caused by skin damage due to ultraviolet ray exposure. Additionally, it was shown that the supernatant of the medium of stem cells stabilized cells and was effective to increase the cell survival rate. When detached from the plate during a passage or divided into single cells, cells suffer physical stresses such as pressure and chemical stresses from the cell detachment agent. However, since cells suffering these stresses grew in the medium containing the supernatant of Reference Example, it was further indicated that the medium containing the supernatant of Reference Example reduced the stresses imposed on cells, protected cells from the stresses, and stabilized cells, thus improving the survival rate. These results indicated that the medium containing the supernatant of Reference Example protected nucleic acids, proteins, protein composites, lipoproteins, ribosomes, and biomembranes contained in cells. Here, biomembranes include cell membranes.

1. A method for providing a cosmetic or a pharmaceutical, comprising providing to a subject a cosmetic or a pharmaceutical comprising a supernatant of a medium of pluripotent stem cells derived from cells donated by the subject.

2. The method according to claim 1, wherein the medium is a medium of the pluripotent stem cells cultured and maintained in an undifferentiated state.

3. The method according to claim 1, wherein the subject who donates the cells and the subject to whom the cosmetic or the pharmaceutical is provided are the same human.

4. The method according to claim 1, wherein the pluripotent stem cells are autologous cells of the subject to whom the cosmetic or the pharmaceutical is provided.

5. The method according to claim 1, further comprising examining an effect of the supernatant of the medium of the pluripotent stem cells on somatic cells derived from the subject.

6. The method according to claim 1, wherein the cosmetic or the pharmaceutical is used for any of retention of skin moisture, amelioration of wrinkles, amelioration of skin turgor, and amelioration of sagging.

7. The method according to claim 1, wherein the cosmetic or the pharmaceutical is an injection.

8. A cosmetic or a pharmaceutical comprising a supernatant of a medium of pluripotent stem cells derived from cells donated by a subject, wherein the cosmetic or the pharmaceutical is provided to the subject.

9. The cosmetic or the pharmaceutical according to claim 8, wherein the medium is a medium of the pluripotent stem cells cultured and maintained in an undifferentiated state.

10. The cosmetic or the pharmaceutical according to claim 8, wherein the subject who donates the cells and the subject to whom the cosmetic or the pharmaceutical is provided are the same human.

11. The cosmetic or the pharmaceutical according to claim 8, wherein the pluripotent stem cells are autologous cells of the subject to whom the cosmetic or the pharmaceutical is provided.

12. The cosmetic or the pharmaceutical according to claim 8, wherein an effect of the supernatant of the medium of the pluripotent stem cells on somatic cells derived from a subject has been examined.

13. The cosmetic or the pharmaceutical according to claim 12, wherein the subject from whom the somatic cells are derived and the subject from whom the pluripotent stem cells are derived are the same human.

14. The cosmetic or the pharmaceutical according to claim 8, wherein the cosmetic or the pharmaceutical is used for any of retention of skin moisture, amelioration of wrinkles, amelioration of skin turgor, and amelioration of sagging.

15. The cosmetic or the pharmaceutical according to claim 8, wherein the cosmetic or the pharmaceutical is an injection.
